(19) United States
(12) Patent Application Publication
(10) Pub. No.: US 2026/0295200 A1
(43) Pub. Date: Oct. 1, 2026

(54) METHOD AND ELECTRONIC DEVICE FOR PROVIDING SLEEP-INDUCING MUSIC ACCORDING TO USER STATE DETERMINED ON BASIS OF BIOSIGNAL

(71) Applicant: EWHA UNIVERSITY - INDUSTRY COLLABORATION FOUNDATION, Seoul (KR)

(72) Inventors: Eunju JEONG, Seoul (KR); Hye Jin SHIN, Seoul (KR); Minjung JI, Seoul (KR)

(21) Appl. No.: 19/480,621

(22) PCT Filed: Apr. 24, 2024
(86) PCT No.: PCT/KR2024/005518

§ 371 Date: Oct. 31, 2025

(30) Foreign Application Priority Data
May 4, 2023 (KR) 10-2023-0058161
Jan. 31, 2024 (KR) 10-2024-0015227

(51) Int. Cl. A61M 21/00; G10H 1/00

(52) U.S. Cl. CPC A61M 21/00; G10H 1/0025; G10H 1/0041; A61M 2021/0027; G10H 2210/111; G10H 2220/371; G10H 2240/121

(57) ABSTRACT

Provided is a method of providing sleep-inducing music to a user by an electronic device. The method may include obtaining a biosignal of the user, identifying a current sleep state of the user based on the obtained biosignal, generating the sleep-inducing music based on the current sleep state of the user, and providing the generated sleep-inducing music to the user.

### TECHNICAL FIELD

The present disclosure relates to a method and an electronic device for determining a state of a user based on a biosignal obtained from the user, generating sleep-inducing music for inducing sleep by stages according to a determined current state of the user, and providing the sleep-inducing music to the user.

### BACKGROUND ART

Recently, a ‘wellness’ trend, which is a way of life to actively create healthy conditions, has socially spread. Wellness, as a composite word of well-being, happiness, and fitness, indicates a socially sound state, as well as in body and mind. In addition, to keep pace with digital generation, data-based healthcare has been actualized. Data-based digital healthcare may be expressed as ‘digital wellness’. In accordance with expansion of digital functions available in daily life, a digital wellness technology to take care of health of body and mind by using information and communications technology (ICT) has been developed.

Sleep helps recovery of physical fatigue and maintenance of biorhythms, and thus is very important for human beings. An appropriate amount of sleep is necessary for normal activities and health. However, many people nowadays are too busy with work or study to have enough sleep. Insufficient sleep may cause decrease in memory or concentration, and may also cause various health problems such as obesity, diabetes, hypertension, heart diseases, decrease in immunity, and the like.

Sleep may be sectionalized into a rapid eye movement (REM) sleep state, in which rapid eye movement occurs, and a non-REM sleep state, in which rapid eye movement does not occur. In the REM sleep state, a human dreams, and in this case, not being able to move, he/she does not actually take any motion in dream. The non-REM sleep state may be sectionalized again into first non-REM sleep, second non-REM sleep, i.e., light-sleep states, and third non-REM sleep, i.e., a deep sleep state. The REM sleep state may occupy about 20% to about 25% of a total sleep time, and the non-REM sleep state may occupy about 75% to about 80% of the total sleep time. The REM sleep state and the non-REM sleep state may be repeated at a cycle of about 90 minutes to about 120 minutes, and about five cycles may be in progress overnight.

The appropriate amount of sleep does not simply indicate a sleep time period. In the REM sleep state, mental fatigue is recovered, and stress is relieved. As operation of controlling mental activities is performed in the REM sleep state, insufficient REM sleep may result in difficulty in emotion control or decrease in memory, thinking, and concentration. In the non-REM sleep state, both a brain and a body take a rest to recover physical fatigue. Lack of non-REM sleep may cause weakened immunity, fatigue of the body, and coarse skin. Therefore, a rate of REM sleep and non-REM sleep has to be maintained to a certain degree to take deep sleep.

Considering importance of deep sleep as described above, a technology capable of assisting deep sleep of users through combination of digital wellness and an ICT technology of a sleep medical field is required.

### DISCLOSURE OF INVENTION

### Technical Problem

According to an embodiment of the present disclosure, an electronic device and a method capable of providing sound suitable for a state of a user and inducing sleep by stages by determining a state of the user through real-time analysis on a biosignal of the user and generating sleep-inducing music to provide to the user based on a current state of the user may be provided.

In addition, according to another embodiment of the present disclosure, an electronic device and a method capable of providing sleep-inducing music personalized for a user and optimizing user experiences by generating sleep-inducing music to provide to the user in consideration of user information such as musical preference, genre preference, tendency, and emotional states of the user may be provided.

Furthermore, according to another embodiment of the present disclosure, an electronic device and a method capable of generating a natural music source and efficiently inducing a sleep state of a user by composing, selecting, or arranging sleep-inducing music to provide to a user in consideration of various musical factors may be provided.

Technical goals to be achieved by the embodiments of the present disclosure are not limited to the technical goals mentioned above, and other technical goals may be derived from the following embodiments.

### Solution to Problem

As a technical solution to achieve the technical goal described above, a method of providing sleep-inducing music by an electronic device may include obtaining a biosignal of a user, identifying a current sleep state of the user based on the obtained biosignal, generating the sleep-inducing music based on the current sleep state of the user, and providing the generated sleep-inducing music to the user.

In an embodiment, the biosignal of the user may include at least one of pieces of information regarding electroencephalography, electrocardiogram, a heart rate, eyeball movements, or breath.

In an embodiment, the current sleep state of the user may be recognized as one of wake, drowsy, rapid eye movement (REM) sleep, first non-REM sleep, second non-REM sleep, or third non-REM sleep.

In an embodiment, the generating of the sleep-inducing music may include identifying a target sleep state of the user and generating the sleep-inducing music based on the current sleep state of the user and the target sleep state of the user.

In an embodiment, the method may further include obtaining user information regarding the user. The generating of the sleep-inducing music may include generating the sleep-inducing music based on the current sleep state of the user and the obtained user information.

In an embodiment, the sleep-inducing music may be generated based on at least one musical factor among volume, tempo, rhythm, melody, harmony, timbre, tonality, or a number of layers.

In an embodiment, the generating of the sleep-inducing music may include composing the sleep-inducing music based on the current sleep state of the user.

In an embodiment, the generating of the sleep-inducing music may include selecting music from a database in which pieces of music pre-generated based on the current sleep state of the user are stored.

In an embodiment, the generating of the sleep-inducing music may further include arranging the selected music based on the current sleep state of the user.

In an embodiment, the generated sleep-inducing music may include a sound effect.

As a technical solution to achieve the aforementioned technical goals, an electronic device configured to provide sleep-inducing music to a user may include a biosignal measurement unit, a storage unit configured to store at least one instruction, at least one processor configured to execute the at least one instruction stored in the storage unit, and an output unit. The at least one processor may be configured to, by executing the at least one instruction, obtain the biosignal of the user through the biosignal measurement unit, identify the current sleep state of the user based on the obtained biosignal, generate the sleep-inducing music based on the current sleep state of the user, and provide the generated sleep-inducing music to the user through the output unit.

In an embodiment, the biosignal measurement unit may include at least one of an EEG sensor, an ECG sensor, a heart rate sensor, an eyeball movement sensor, a PPG sensor, an inertial measurement unit (IMU), a camera, or a microphone.

In an embodiment, the output unit may include at least one of a speaker, an earphone, a headset, or a vibration-type earphone.

In an embodiment, the current sleep state of the user may be identified as wake, drowsy, rapid eye movement (REM) sleep, first Non-REM (NREM) sleep, second NREM sleep, or third NREM sleep.

In an embodiment, the electronic device may further include a user input receiver.

In an embodiment, the at least one processor may be configured to obtain information regarding the target sleep state of the user through the user input receiver and generate the sleep-inducing music based on the current sleep state of the user and the target sleep state of the user.

In an embodiment, the at least one processor may obtain user information regarding the user through the user input receiver and generate the sleep-inducing music based on the current sleep state of the user and the obtained user information.

In an embodiment, the at least one processor may generate the sleep-inducing music based on at least one musical factor among volume, tempo, rhythm, melody, harmony, timbre, tonality, or the number of layers.

In an embodiment, the storage unit may include a database in which pieces of pre-generated music are stored. The at least one processor may select music from the data based on the current sleep state of the user, and arrange the selected music based on the current sleep state of the user to generate the sleep-inducing music.

In an embodiment, the generated sleep-inducing music may include a sound effect.

As a technical solution to achieve the aforementioned technical goal, a computer-readable recording medium may store a program for executing, on a computer, at least one of embodiments of the disclosed method.

### Advantageous Effects of Invention

According to a method and a device for providing sleep-inducing music according to a user state determined according to a biosignal, as suggested in the present disclosure, by determining a state of the user based on real-time analysis on a biosignal of a user and generating the sleep-inducing music to be provided to the user based on a current state of the user, sound suitable for the user state may be provided, and the user may be induced by stages to sleep.

In addition, according to a method and a device for providing sleep-inducing music according to a user state determined according to a biosignal, as suggested in the present disclosure, by generating sleep-inducing music to provide to the user in consideration of user information such as musical preference, genre preference, personality, and emotional states of the user, personalized sleep-inducing music may be provided to the user, and user experiences may be optimized.

Furthermore, according to a method and a device for providing sleep-inducing music according to a user state determined according to a biosignal, as suggested in the present disclosure, by composing, selecting, or arranging the sleep-inducing music to provide to the user in consideration of various musical factors, natural musical sources may be generated, and a sleep state of the user may be efficiently induced.

### BRIEF DESCRIPTION OF DRAWINGS

FIG. 1 is a flowchart of a method of providing sleep-inducing music, by an electronic device according to an embodiment of the present disclosure, according to a state of a user determined based on a biometric signal;

FIG. 2A is a diagram for describing an emotional state of a user according to an embodiment of the present disclosure;

FIG. 2B is a diagram for describing an operation of determining timbre to be used for the sleep-inducing music in consideration of an emotional state of a user according to an embodiment of the present disclosure;

FIG. 3 is a diagram for describing an operation of providing sleep-inducing music by stages according to a sleep state of a user by an electronic device according to an embodiment of the present disclosure;

FIG. 4 is a diagram for describing an operation of providing sleep-inducing music, by an electronic device according to an embodiment of the present disclosure, to induce a user in a wake state to a sleep state;

FIG. 5 is a diagram for describing an operation of providing sleep-inducing music, by an electronic device according to an embodiment of the present disclosure, to maintain a sleep state of a user;

FIG. 6 is a diagram for describing an operation of providing sleep-inducing music, by an electronic device according to an embodiment of the present disclosure, to induce a user in a sleep state to a wake state; and

FIG. 7 is a block diagram of an electronic device according to an embodiment.

### MODE FOR THE INVENTION

Hereinafter, embodiments of the present disclosure will be described in detail with reference to the accompanying drawings for those skilled in the art of the present disclosure, to easily implement the embodiments. However, the present disclosure may be implemented in various different forms and is not limited to embodiments described herein. For clear explanation of the present disclosure, parts of the drawings irrelevant to the descriptions were omitted, and throughout the specification, similar reference numerals have been used for similar components.

Although general terms widely used nowadays have been selected as the terms used in the present disclosure in consideration of functions in the disclosure, the terms may vary according to intention of those skilled in the art, judicial precedents, and emergence of new technologies. Furthermore, in specific occasions, terms arbitrarily selected by the applicant may also be used, and in this case, meanings thereof will be written in detail in descriptions of corresponding embodiments. Therefore, terms used in the present specification will be used based on meanings of the terms and contents throughout the present disclosure, not based on mere names of the terms.

Unless explicitly indicated otherwise in the context, singular forms may encompass plural forms. Terms used herein, including technical or scientific terms, may have same meanings as meanings generally understood by those skilled in the art disclosed in the present specification.

Throughout the present disclosure, unless particularly disclosed otherwise, the expression that a portion “includes” a component indicates that the portion may further include another component, not that the other component is excluded. In additions, terms such as “~part”, “~module” written in the present specification indicates a unit configured to process at least one function or operation, and may be implemented by hardware, software, or combination of hardware and software.

Throughout the specification, expression that a portion is “connected” to another portion includes a case where the portion and the other portion are “electrically connected to each other” having an element therebetween, as well as case where the portion and the other portion are “directly connected to each other”. In addition, unless particularly disclosed otherwise, the expression that a portion “includes” a component indicates that the portion may further include another component, not that the other component is excluded.

In some cases, expression “configured to” used in the present specification may be compatible with, for example, “suitable for”, “having the capacity to”, “designed to”, “adapted to”, “made to”, or “capable of”. The term “configured to” may not necessarily and exclusively indicate “specifically designed to” in terms of hardware. Instead, in some occasions, the expression “a system configured to~” may indicate that the system may be “configured to~” together with other devices or components. For example, a phrase “a processor configured to perform A, B, and C” may indicate a dedicated processor (e.g., an embedded processor) configured to perform corresponding operations or a generic-purpose processor (e.g., a central processing unit (CPU) or an application processor) capable of performing corresponding operations by executing one or more software programs stored in a memory.

Artificial intelligence (AI)-related functions according to the present disclosure will be operated through a processor and a memory. The processor may include one processor or a plurality of processors. In this case, the one or more processors may include a generic-purpose processor such as a CPU, an Application Processor (AP), a Digital Signal Processor (DSP), a graphic-dedicated processor such as Graphic Processing Unit (GPU), a Vision Processing Unit (VPU), or an AI-dedicated processor such as NPU. The one or more processors control processing of input data according to predetermined operation rules or AI models stored in the memory. When the one or more processors include an AI-dedicated processor, the AI-dedicated processor may be designed in a hardware structure specialized for processing particular AI models.

The predetermined operation protocols or AI models are constructed through learning. Here, construction through learning indicates that a basic artificial intelligence model (or a deep learning model) is trained by using pieces of learning data through based on a learning algorithm and thus a pre-defined operation rule or artificial intelligence model set to perform desired properties (or purposes) is constructed. Such training may be performed in a device itself configured to perform artificial intelligence according to the present disclosure, and may also be performed through an additional server and/or a system. Examples of the learning algorithms include supervised learning, unsupervised learning, semi-supervised learning, or reinforcement learning, but are not limited thereto.

The artificial intelligence model (or the deep learning model) may include a plurality of neural network layers. Each of the plurality of neural network layers has a plurality of weight values, and is configured to perform neural network operation through operation between an operation result from a previous layer and the plurality of weight values. The plurality of weight values held by the plurality of neural network layers may be optimized based on a result of training of the artificial intelligence model. For example, the plurality of weight values may be updated such that a loss value or cost value obtained from the artificial intelligence model during the training is reduced or minimized. An artificial neural network may include Deep Neural Network (DNN), and may include, for example, Convolutional Neural Network (CNN), Recurrent Neural Network (RNN), Restricted Boltzmann Machine (RBM), Deep Belief Network (DBN), Bidirectional Recurrent Deep Neural Network (BRDNN), Deep Q-Network, or the like, but is not limited to the aforementioned examples.

In the present disclosure, ‘sleep’ may indicate that a human sleeps. A sleep state may be sectionalized into a rapid eye movement (REM) sleep state, in which rapid eye movement occurs, and a Non-REM (NREM) state, in which rapid eye movement does not occur. The NREM state may be sectionalized in detail into first NREM, second NREM, i.e., light-sleep states, and third NREM, i.e., a deep-sleep state. In the present disclosure, change in the sleep state ‘by stages’ may indicate change by at least one stage from a REM state, first NREM, second NREM, and third NREM.

In the present disclosure, ‘deep sleep’ may indicate high-quality sleeping. For example, sleep for a total time period within an appropriate range and having a ratio of a time period in the REM state and a time period in the NREM state in the entire sleeping time period within a certain range may be deep sleep. When the ratio of the time period in the REM state and the time period in the NREM state in the entire sleeping time period is within the certain range, mental fatigue and physical fatigue may be appropriately recovered, and high-quality sleep may be realized.

In the present disclosure, ‘musical elements’ may indicate characteristics of capable of distinguishing music. The musical elements include volume, tempo, rhythm, melody, harmony, timbre, tonality, or the number of layers.

The volume indicates intensity of the music. Difference in volume may be distinguished by sound of music being ‘loud/quiet’. The tempo indicates speed of the music. Difference in the tempo may be distinguished by the speed of the music (e.g., beats per minute (bpm)) being ‘fast/slow’. The rhythm indicates a speed pattern of music as time passes. The melody, which is a group of notes arranged in horizontal or sequential orders, indicates a height pattern of music as time passes. The harmony indicates a pattern made by continuous chords according to a time order.

Pitch (or Scale) indicates height of sound. The pitch may be marked with Hz unit that indicates the number of vibration of waveform per second. A greater number of vibrations per second indicates higher pitch, and when the number of vibrations is twice, a note above an octave is generated. The pitch may indicate an absolute height of the note, and the scale may indicate a relative height of the note. For example, the pitch may indicate a note in an octave of a chromatic scale to which a certain note corresponds, and the scale may indicate, through numbering, the relative height of the note according to harmony.

The timbre indicates an inherent color of note of sound. The timbre may differ depending on sound sources. For example, when music having same tempo, rhythm, melody, harmony, and tonality is played by piano and violin, the timbre may differ and thus may be distinguished.

The tonality indicates predetermined certain chord rules. Music with tonality may indicate that the harmony or melody used for the music is under certain rules. The tonality may be distinguished according to identity or difference of the rules.

Layers may be divided according to a texture or structural properties of music. The texture may be distinguished based on, for example, whether the music belongs to monophony or belongs to polyphony, homophony, or heterophony. The structure may be distinguished according to flow of the entire music in consideration of an intro, a verse, a chorus, an interlude, a bridge, and an outro. The number of layers indicates the number of sound sources overlapping on a time axis.

Hereinafter, a method of generating sleep-inducing music according to a state of a user determined based on a biosignal and providing the sleep-inducing music to the user and an electronic device therefor, according to an embodiment of the present disclosure, will be described in detail.

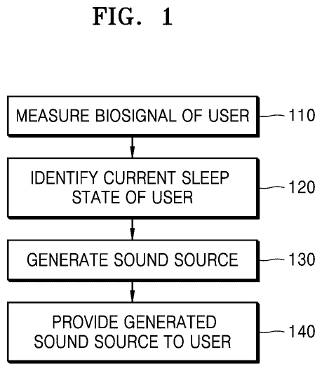

FIG. 1 is a flowchart of a method of providing sleep-inducing music, by an electronic device according to an embodiment of the present disclosure, according to a user condition determined based on a biosignal.

In operation 110, the electronic device may obtain a biosignal of the user. For example, the biosignal of the user may be measured through a biosignal measuring unit included in the electronic device.

For example, the biosignal of the user may include at least one of information regarding electroencephalography (EEG), electrocardiogram (ECG), a heart rate, eyeball movements or breaths. The biosignal of the user are not limited to the aforementioned examples.

A brain wave or EEG indicates flow of electricity generated when information (signal) is delivered between cranial nerves in a human nervous system. The brain wave that may be obtained through a non-invasive method may be measured through electrodes attached to a scalp without additional surgical operations, and enables tracking for real-time brain operation. The EEG may indicate a technology of attaching an electrode to a cortical substance and recording the brain wave, and may also indicate a graphic expression of the brain wave recorded through such technology. The brain wave may have a waveform vibrating in a complex pattern. A power spectrum analysis method to categorize the brain wave according to frequencies may be used for brain wave analysis. The power spectrum analysis method may be performed by assuming that the brainwave is a linear combination of vibrations having particular frequencies and decomposing each frequency component to calculate size thereof. A brainwave generated in a human brain may have radiofrequency about 0 Hz to about 50 Hz and an amplitude about 20 μV to about 200 μV. The EEG may be measured through an EEG sensor. The brain wave may vary depending on a sleep state of a human. Accordingly, a current sleep state of the user may be recognized by measuring the EEG.

The ECG is a record about electrical movement of a heart. The ECG may be measured through electrodes attached to a body. The ECG may be used for measuring a ratio and consistency of heart beats. The ECG may be measured through an ECG sensor. The heart rate indicates the number of heart beats per unit time. The heart rate may be indicated as beats per minutes (bpm). The heart rate may be measured through a heart rate sensor, an ECG sensor, photoplethysmography (PPG), a camera, or a microphone. When a user in a comfortable state, the heart rate may decrease. Accordingly, the heart rate may be used for an operation to distinguish whether the user is drowsy, or is waken, or in the REM sleep state with dreams, or in a deep-sleep state.

The eyeball movement may be used for an operation to measure whether a current sleep state of the user is the REM sleep state. The eyeball movement may be measured by determining whether eyelids of the user shake in a video obtained through the camera.

Information regarding breath may include the number of breath, an amount of breath, or status of snoring. In an embodiment, the information regarding breath may be used for an operation to distinguish whether the user is drowsy or waken, or whether the user is in sleep or is waken.

In operation 120, the electronic device may recognize the current sleep state of the user based on the obtained biosignal of user.

In an embodiment, the current sleep state of the user may be recognized as one of wake, drowsy, rapid eye movement (REM), the first NREM sleep Non REM 1, the second NREM sleep Non REM 2, or the third NREM sleep Non REM 3.

The sleep state corresponds to a deeper sleep state from wake, drowsy, rapid eye movement (REM) sleep, the first NREM sleep Non REM 1, the second NREM sleep Non REM 2, and the third NREM sleep Non REM 3.

In operation 130, the electronic device may generate sleep-inducing music based on the current sleep state of the user.

The electronic device may be configured to generate the sleep-inducing music based on at least one of musical factors from among volume, tempo, rhythm, melody, harmony, timbre, tonality, or the number of layers. In an embodiment, the electronic device may be configured to composition to generate the sleep-inducing music based on the current sleep state of the user.

Here, a method of determining each music factor in generation of the sleep-inducing music based on the current sleep state of the user will be described.

The volume may be highest when the sleep state of the user is the wake state and lowest when the sleep state of the user is the third NREM sleep state. That is, as the sleep state of the user progresses to the deep sleep state, the volume may be lowered. In an embodiment, when the user is in the third NREM sleep state taking deepest sleep, the volume may be determined not to exceed about 30 dB. In an embodiment, the volume when the sleep state of the user is the wake state may also be determined based on selection of the user. Even when the user selects a volume in the wake state, the volume of the sleep-inducing music thereafter may be adjusted according to the sleep state of the user.

The rhythm may match a brainwave according to the sleep state of the user. For example, the brainwave of the user may correspond to a Beta wave in the wake state, correspond to an Alpha wave in the drowsy state or the REM sleep state, correspond to a Theta wave (V wave, K complex & Spindel) in the first or second NREM sleep state, and correspond to a Delta wave in the third NREM sleep state. In an embodiment, a relatively fast tempo (or a rhythm having same intervals) may match vibrations in a Theta band, and a relatively slow tempo (or a rhythm having same intervals) may match vibrations in a Delta band. Accordingly, the rhythm may be fastest when the sleep state of the user is the wake state, and may be slowest whether the sleep state of the user is the third NREM sleep state. As the sleep state of the user progresses from the wake state (or light sleep) to the deep sleep state, the rhythm may become slower. In an embodiment, when the user is in the third NREM sleep state taking deepest sleep, the tempo may be determined to be a slow wave of about 2 Hz or less.

In an embodiment, the tempo of the sleep-inducing music provided to the user may be determined based on selection of the user. The user may select the tempo of the sleep-inducing music from an area about 60 bpm to about 120 bpm. When the user selects the tempo of the sleep-inducing music, a rhythm when the user is in the wake state (an initial rhythm) may be determined based on the tempo selected by the user. In an embodiment, once the user selects the tempo (the initial rhythm), a phonetic value of the rhythm of the sleep-inducing music changes according to the sleep state of the user to induce the user to be in the sleep state. The phonetic value of the rhythm of the sleep-inducing music may be least when the sleep state of the user is the wake state and greatest when the sleep state of the user is the third NREM sleep state. For example, the tempo selected by the user may be used for an operation to determine a rhythm of the sleep-inducing music provided when the sleep state of the user is the wake state. Thereafter, as the user progresses to a deeper sleep state, the phonetic value of the rhythm of the sleep-inducing music increases, and thus the user may feel that the sleep-inducing music is provided at a slower tempo. That is, as the sleep state of the user progresses from the wake state (or light sleep) to the deep sleep state, the phonetic value of the rhythm may increase. In an embodiment, when the user is in the third stage Non-REM sleep state taking deepest sleep, the sleep-inducing music may be determined to have a slow wave of about 2 Hz or less. For example, sleep-inducing music composed of quarter notes or eighth notes, which is of about 90 bpm, may effectively induce relaxing of the user.

The harmony may be highly complex when the sleep state of the user is the wake state, and may be less complex when the sleep state of the user is the third NREM sleep state. That is, as the sleep state of the user progresses from the wake state (or light sleep) to the deep sleep state, the complexity may be reduced. In an embodiment, the harmony may also be determined based on user information such as musical preference of the user. For example, when the user particularly prefers a certain harmony, the harmony may be used in the sleep-inducing music. For example, the harmony that the user particularly prefers may be used as harmony of the sleep-inducing music that is provided when the sleep state of the user is the wake state. Thereafter, as the user progresses to deeper sleep states, the harmony of the sleep-inducing music may have less complexity.

The melody may have greatest change in tunes when the sleep state of the user is the wake state, and may have least change in tunes when the sleep state of the user is the third NREM sleep state. In addition, as the sleep state of the user progresses from the wake state (or the light sleep) to deeper sleep states, degree of recognition of initial melody of the sleep-inducing music may decrease. That is, as the sleep state of the user progresses from the wake state (or the light sleep) to the deeper sleep states, a profile of the initial melody may be gradually removed.

In an embodiment, the electronic device may be configured to identify a target sleep state of the user and generate the sleep-inducing music further based on the target sleep state. For example, the target sleep state at a certain time point may be determined in consideration of a lasting time period of the current sleep state at the corresponding time point and a lasting time period of a previous sleep state. For example, when the target sleep state of the user is the ‘deep sleep state’ and the current sleep state of the user is the ‘wake state’, ‘the drowsy state’ (sleep onset induced), or the ‘light-sleep state’ (deep-sleep maintained), the electronic device may generate the sleep-inducing music for inducing the user by stages from the current state to the deep sleep state. For example, when the target sleep state of the user is the ‘wake state’ and the current sleep state of the user is the ‘light sleep state’ or the ‘deep sleep state’ (awake-inducing), the electronic device may generate the sleep-inducing music for inducing the user by stages from the current state to the wake state.

In an embodiment, the electronic device may obtain user information including musical preference (player, instrumental composition, and the like), tendency, and emotional state of the user, and may generate sleep-inducing music personalized for the user, further based on the user information. For example, the electronic device may generate the sleep-inducing music by using timbre or tonality matching an emotional state desired by the user, or may generate the sleep-inducing music to include an environmental sound matching a certain emotional state. The user information may be pre-stored in the electronic device, or may be received through a user input receiver included in the electronic device.

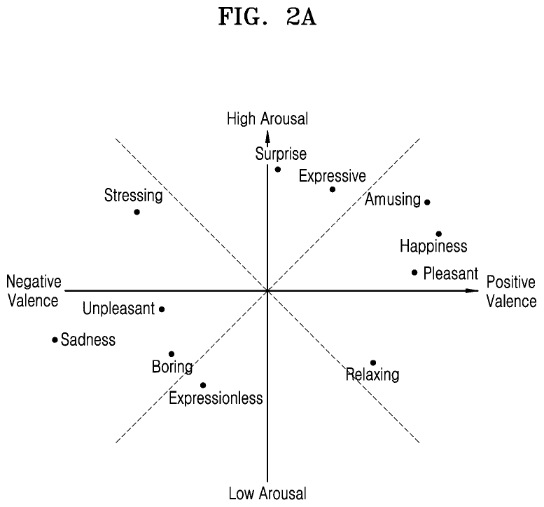

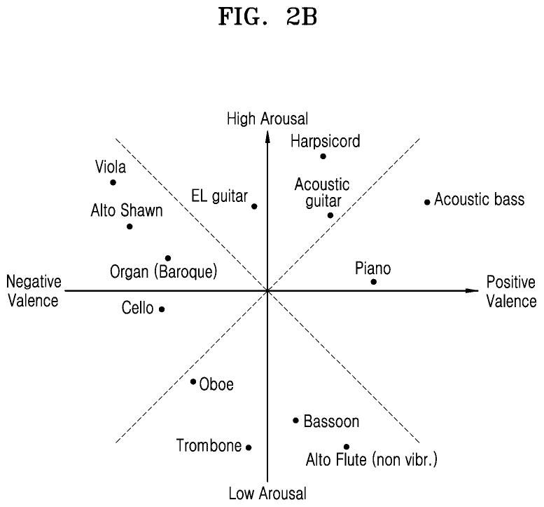

For example, the electronic device may differently map musical instruments used for generating the sleep-inducing music according to the emotional state of the user included in the user information. Different musical instruments have different timbres. Matching tendencies or emotions may vary according to timbres. For example, brass instruments or high-pitched string instruments may be suitable for outgoing tendency or emotional states of high arousal (e.g., elation, joy, fun, anger, and the like), tune percussions or keyboard instruments may be suitable for emotional states of medium arousal (e.g., joy, happiness, and the like), and wind instruments may be suitable for introverted tendency or emotional states of low arousal (e.g., sadness, languor, boredom, and the like). The electronic device according to an embodiment of the present disclosure may determine a musical instrument (timbre) to be included in generation of the sleep-inducing music based on the user information such as the tendency or emotional state of the user. In an embodiment of the present disclosure, an operation of selecting the musical instrument based on the user information including the emotional state of the user and generating the sleep-inducing music personalized for the user will be described in further detail with reference to FIGS. 2A and 2B to be described hereinafter.

In an embodiment, the electronic device may also generate the sleep-inducing music personalized for the user, based on selection of the user. For example, the tempo of the sleep-inducing music provided to the user may be determined based on the selection of the user. The user may select the tempo of the sleep—inducing music from an area about 60 bpm to about 120 bpm.

According to an embodiment of the present disclosure, the electronic device may generate the sleep-inducing music based on the current sleep state of the user identified from the biosignal of the user, and according to embodiments, may generate the sleep-inducing music personalized for the user to be provided to the user, based on the user information in addition to the current sleep state of the user. Various musical factors such as volume, tempo, rhythm, melody, harmony, timbre, tonality, the number of layers, and the like may be considered in generation of the sleep-inducing music according to the present disclosure. That is, the electronic device may determine each of various musical factors of the sleep-inducing music, based on the current sleep state of the user and the user information.

For example, the musical factors such as the volume, the rhythm, the melody, the harmony, or the number of layers according to a time period of the sleep-inducing music are determined real-time based on the current sleep state of the user, and the timbre, the tonality, or musical factors of the environmental sound selectively included may be previously determined based on the user information such as the preference or the emotional state of the user. In addition, musical factors such as an initial volume or tempo of the sleep-inducing music may be previously determined based on the selection of the user from among the ‘user information’. That is, according to an embodiment of the present disclosure, in generating the sleep-inducing music, may generate personalized music based on the user information such as the preference of the user, the emotional state of the user, or the selection of the user, and may provide sound suitable for the state of the user and efficiently induce sleep of the user by adjusting the musical factors in real-time based on the current sleep state of the user.

In an embodiment, the electronic device may also select music from a database in which previously generated pieces of music are stored, based on the current sleep state of the user. The database may be stored in a storage unit of the electronic device. In an embodiment, the electronic device may also arrange the music selected from the database, based on the current sleep state of the user. The arrangement may be performed in a method of modifying at least one musical factor of the selected music. For example, the electronic device may arrange the music by modifying at least one of the volume, the tempo, the rhythm, the melody, the harmony, the timbre, or the tonality of the selected music, and determine the arranged music as the sleep-inducing music.

In an embodiment, the sleep-inducing music generated by the electronic device may include sound effects. For example, the sound effects may include sound of waves, sound of water, sound of leaves trembling in wind, sound of wind, sound of birds, sound of laughter. In an embodiment, the sleep-inducing music may be composed of at least one sound effect, may be composed of a combination of at least one sound effect and music, or may be composed only of music. For example, a type of sound effect included in the sleep-inducing music may be determined based on the user information such as the emotional state of the user. An operation of selecting a sound effect to be included in the sleep-inducing music based on the user information will be described in further detail with reference to FIG. 2A to be described hereinafter.

In operation 140, the electronic device may provide the generated sleep-inducing music to the user. The generated sleep-inducing music may be provided to the user through an output unit included in the electronic device.

FIG. 2A is a diagram for describing the emotional state of the user according to an embodiment of the present disclosure, and FIG. 2B is a diagram for describing an operation of determining timbre to be used for the sleep-inducing music in consideration of the emotional state of the user according to an embodiment of the present disclosure.

In an embodiment, the electronic device may obtain user information including the musical preference (player, instrumental composition, and the like), the tendency, and the emotional state of the user, and may generate sleep-inducing music personalized for the user, further based on the user information. For example, the musical factor adjusted based on the user information may be the timbre or the tonality, and type of the sound effect selectively included in the sleep-inducing music may also be determined based on the user information. For example, the electronic device may generate the sleep-inducing music by using timbre or tonality matching an emotional state desired by the user, or may generate the sleep-inducing music to include an environmental sound matching a certain emotional state. The user information may be pre-stored in the electronic device, or may be received through a user input receiver included in the electronic device.

Referring to FIG. 2, the emotional state of the user may be indicated as a quadrant of emotion. In the quadrant of emotion, an x axis indicates a valence. A higher valence corresponds to a more positive emotion, and a lower valence corresponds to a more negative emotion. In the quadrant of emotion, a y axis indicates degree of arousal (or degree of excitement). In an embodiment, the musical factor such as the timbre or the tonality of the sleep-inducing music to be provided to the user and the environmental sound to be included in the sleep-inducing music may be determined based on the emotional state desired by the user.

Referring to FIG. 2A, the emotional state of the user may be roughly sectionalized into eight states. In a first quadrant, an area where the degree of arousal is more significant than the valence may be ‘Aroused/Excited’ area, which may include ‘Surprise’ state and ‘Expressive’ state. In the first quadrant, an area where the valence is more significant than the degree of arousal may be ‘Happy/Delighted’ area, which may include ‘Amusing’ state, ‘Happiness’ state, and ‘Pleasant’ state. In a second quadrant, an area where the degree of arousal is more significant than the valence may be ‘Angry/Afraid’ area. In the second quadrant, an area where the valence is more significant than the degree of arousal may be ‘Distressed/Frustrated’ area, which may include ‘Stressing’ state. In a third quadrant, an area where the degree of arousal is more significant than the valence may be ‘Tired/Bored’ area, which may include ‘Expressionless’ state. In the third quadrant, an area where the valence is more significant than the degree of arousal may be ‘Miserable/Sad’ area, which may include ‘Unpleasant’ state, ‘Sadness’ state, and ‘Boring’ state. In a fourth quadrant, an area where the degree of arousal is more significant than the valence may be ‘Sleepy/Peaceful’ area. In the fourth quadrant, an area where the valence is more significant than the degree of arousal may be ‘Pleased/Relaxed’ area, which may include ‘Relaxing’ state. The emotional state of the user is not limited to the examples mentioned above, and various emotional states having various degree of wakening and valences may be set.

In an embodiment, musical instruments to be used for generating the sleep-inducing music may be differently matched according to the emotional state of the user. Different musical instruments have different timbres. Matching tendencies or emotions may vary according to timbres.

In an embodiment, suitable timbres may be determined according to the emotional state of the user determined based on the degree of arousal and the valence. For example, brass instruments or high-pitched string instruments may be suitable for emotional states with high arousal (e.g., elation, joy, fun, anger, and the like), tune percussions or keyboard instruments may be suitable for emotional states with middle arousal (e.g., joy, happiness, and the like), and wind instruments may be suitable for emotional states with low arousal (e.g., sadness, languor, boredom, and the like).

Referring to FIG. 2B, a timbre of a harpsichord or an acoustic guitar may be suitable for an emotional state in the ‘Aroused/Excited’ area in FIG. 2A, and a timbre of an acoustic bass or a piano may be suitable for an emotional state in the ‘Happy/Delighted’ area. A timbre of an electric guitar may be suitable for an emotional state in the ‘Angry/Afraid’ area, and a timbre of a viola, an alto Shawn, or a baroque organ may be suitable for an emotional state in the ‘Distressed/Frustrated’ area. A timbre of an oboe or a trombone may be suitable for an emotional state of the ‘Tired/Board’ area, and a timbre of a cello may be suitable for an emotional state of the “Miserable/Sad’ area. A timbre of a bassoon or an alto flute may be suitable for an emotional state in the ‘Sleepy/Peaceful’ area. Like this, the electronic device according to an embodiment of the present disclosure may determine musical instruments (timbres) to be included in generation of the sleep-inducing music, based on the emotional state of the user. The timbres suitable for the emotional states of the user described with reference to FIG. 2B are not limited to the examples described above, and various musical instruments (timbres) may be matched for various emotional states.

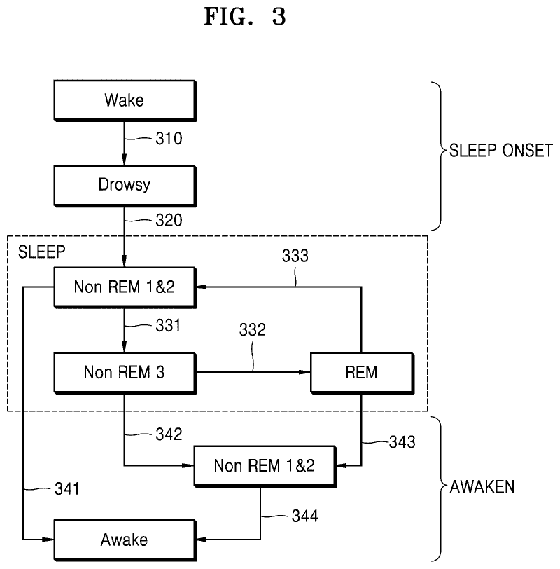

FIG. 3 is a diagram for describing an operation of inducing the sleep state of the user by adjusting the musical factors, the operation in which the electronic device according to an embodiment of the present disclosure provides the sleep-inducing music by stages according to the sleep state of the user.

Referring to FIG. 3, the sleep period of the user may be roughly sectionalized into a sleep onset stage, a sleep state, and an awake stage. The sleep onset stage may indicate a section in which the user starts from the wake state, goes through the drowsy state, and then enters the first or second NREM sleep state Non REM 1 and 2, i.e., the light sleep state. In the sleeping stage, the sleep state of the user may be one of the first or second NREM sleep state Non REM 1&2, the third NREM sleep state Non REM 3, and the REM sleep state. In the sleeping stage, the first or second NREM sleep state Non REM 2, the third NREM sleep state Non REM 3, and the REM sleep state may be repeated. The Awake stage may indicate a section in which the user is out of the sleep state and enters the Awake state.

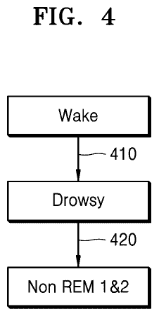

In a sleep onset-inducing stage, the electronic device may provide the sleep-inducing music to induce the user to the Drowsy state, i.e., the target state, when the current state of the user is the Wake state (operation 310). Next, when it is determined that the current state of the user progressed to the Drowsy state, the electronic device may provide the sleep-inducing music to induce the state of the user from the Drowsy state to the first or second NREM sleep Non REM 2, i.e., the target state at a next stage (operation 320). The sleep-inducing music provided to induce the user to the sleep onset will be described in further detail with reference to FIG. 4 to be described hereinafter.

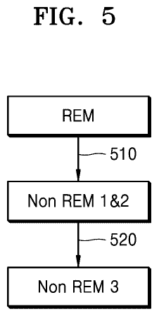

In a sleep maintenance stage, the electric device may provide the sleep-inducing music for progress to the next stage, according to the current sleep state of the user. In the sleep stage, the sleep state of the user may progress from the first or second NREM sleep Non REM 1&2 to the third NREM sleep Non REM 3 (operation 331), progress from the third NREM sleep Non REM 3 state to the REM sleep state (operation 332), and may progress from the REM sleep state REM back to the first or second NREM sleep Non REM 2 (operation 333). The first or second NREM sleep state Non REM 1&2, the third NREM sleep state Non REM 3, and the REM sleep state REM may be repeated about four times within a sleep cycle. The sleep-inducing music provided for maintaining the sleep of the user will be described in further detail with reference to FIG. 5 to be described hereinafter.

In an awake-inducing stage, the electronic device may provide sleep-inducing music for inducing the user from the current sleep state to the Wake state. At a time point of awake-inducing, the sleep state of the user may be the first or second NREM sleep Non REM 1&2, the third NREM sleep Non REM 3 state, or the REM sleep state REM. Herein, when the user is wake-induced in the first or second NREM sleep Non REM 1&2, the electronic device may provide sleep-inducing music for the user to immediately progress to the awake state.

When the user at the time point of awake-inducing is in the third NREM sleep state Non REM 3 or the REM sleep state REM, having the user progress to the wake state right away may cause severe fatigue or confusion of the user. Therefore, the user may be first induced from the third NREM sleep state Non REM 3 to the first or second NREM sleep state Non REM 1&2 (operation 342) and then induced to the wake state (operation 344), or may be first induced from the REM sleep state REM to the first or second NREM sleep state Non REM 1&2 (operation 343) and then to the wake state (operation 344).

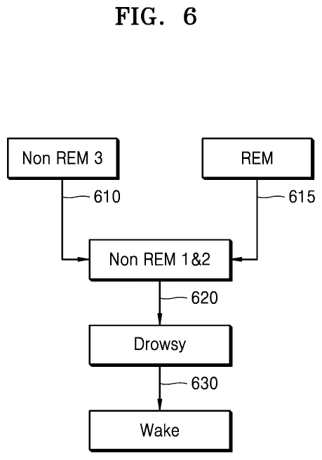

The sleep-inducing music provided to induce the user to be awake will be described in further detail with reference to FIG. 6 to be described hereinafter.

FIG. 4 is a diagram for describing an operation of providing sleep-inducing music, by an electronic device according to an embodiment of the present disclosure, to induce the user in the wake state to the sleep states.

Operations in FIG. 4 may be applied to induce the user to the sleep onset state.

In operation 410, the electronic device may provide the sleep-inducing music to induce the user to the Drowsy state, i.e., the target state, when the current state of the user is the wake state. The sleep-inducing music provided in operation 410 is to provide a comfortable atmosphere to induce the user to the sleep state. When providing music for the user in the wake state to be initially induced to the sleep onset, sleep-inducing music, to which the user information including the musical preference of the user is preferentially reflected, may be provided. Operation 410 may correspond to operation 310 in FIG. 3 described above.

In operation 420, when it is determined that the current state of the user progressed to the Drowsy state, the electronic device may provide the sleep-inducing music to induce the state of the user from the Drowsy state to the first or second NREM sleep state Non REM 1&2, i.e., the target state at the next stage. Compared with the sleep-inducing music provided in operation 410, the sleep-inducing music provided in operation 420 may have less melody, slower tempo, slower rhythm, or reduced complexity of harmony. Operation 420 may correspond to operation 320 in FIG. 3 described above.

In a process where the sleep-inducing music provided in operation 410 is updated to the sleep-inducing music provided in operation 420, the melody may be first removed, and then the rhythm may slow down, and last, the complexity of harmony may be reduced, but the embodiment is not limited thereto. The volume may linearly decrease for a certain time period while progressing from operation 410 to operation 420.

FIG. 5 is a diagram for describing an operation of providing sleep-inducing music, by an electronic device according to an embodiment of the present disclosure, to maintain the sleeping state of the user.

Operation in FIG. 5 may be applied to maintain the sleep of the user. In a sleep cycle, a cyclic operation may be repeated about four times, in which the sleep state of the user progresses from the first or second NREM sleep state Non REM 1&2 to the third NREM sleep state Non REM 3, progresses to the REM sleep state REM, and then again to the first or second NREM sleep state Non REM 1&2. ‘Sleep maintenance’ may indicate having the user sequentially progress to the sleeping states according to the cyclic operation.

That is, the sleep maintenance is not limited to the following example, and may include inducing the sleep state of the user from the first or second NREM sleep state Non REM 1&2 to the third NREM sleep state Non REM3, inducing the sleep state of the user from the first or second NREM sleep state Non REM 1&2 to the REM sleep state REM, inducing the sleep state of the user from the third NREM sleep state Non REM 3 to the first or second NREM sleep state Non REM 1&2, inducing the sleep state of the user from the REM sleep state REM to the first NREM or second NREM sleep state Non REM 1&2 to the third Non-REM sleep state Non REM 3.

In operation 510, the electronic device may provide sleep-inducing music to induce the sleep state of the user to the first or second NREM sleep state Non REM 1&2 when the current state of the user is the REM sleep state REM. The sleep-inducing music provided in operation 510 is to induce the user again to the Non-REM sleep state after occurrence of the REM sleep state while sleeping. Operation 510 may correspond to operation 333 in FIG. 3 described above.

In operation 520, when it is determined that the current state of the user changes to the first or second NREM sleep state Non REM 2, the electronic device may provide sleep-inducing music to induce the state of the user from the first or second NREM sleep state Non REM 1&2 to the third NREM sleep state Non REM 3, i.e. the target state in a next stage. The sleep-inducing music provided in operation 520 is to induce the user to a deeper sleep state. Compared with the sleep-inducing music provided in operation 510, the sleep-inducing music provided in operation 520 may have slower tempo, slower rhythm, or reduced complexity of harmony. Operation 520 may correspond to operation 331 in FIG. 3 described above.

In a process where the sleep-inducing music provided in operation 510 is updated to the sleep-inducing music provided in operation 520, the melody may be first removed, and then the rhythm may slow down, and last, the complexity of harmony may be reduced, but the embodiment is not limited thereto. The volume may linearly decrease for a certain time period while progressing from operation 510 to operation 520.

FIG. 6 is a diagram for describing an operation of providing sleep-inducing music, by an electronic device according to an embodiment of the present disclosure, to induce a user in a sleeping state to a wake state.

Operation in FIG. 6 may be applied to induce the user to be awake from the sleep state. At the time point of awake-inducing, the sleep state of the user may be the first or second NREM sleep Non REM 1&2, the third NREM sleep Non REM 3 state, or the REM sleep state REM.

Here, when the user is induced to be awake in the first or second NREM sleep state Non REM 1&2, the electronic device may progress to operation 620 and provide the sleep-inducing music to induce the user to immediately progress to the Awake state.

Transferring (awakening) from the third Non-REM sleep state Non REM 3 right to the Wake state may cause severe fatigue to the user. Therefore, it is desirable to awake while transferring by stages between the sleep states, and more particularly, it is desirable to first transfer from the third NREM sleep state Non REM 3 to the first or second Non-REM sleep state Non REM 1&2 (operation 610) and then transfer to the Wake state. Operation 610 may correspond to operation 342 in FIG. 3 described above.

The REM sleep state is a dreaming state or is related to strong emotional experiences, therefore, transferring (awakening) from the REM sleep state REM right to the Wake state may make the user feel confused. Therefore, also in this case, it is desirable for the user to awake while transferring by stages between the sleep states, and more particularly, it is desirable to first transfer from the REM sleep state (REM) to the first or second NREM sleep state Non REM 1&2 (operation 615) and then transfer to the Wake state. Operation 615 may correspond to operation 343 in FIG. 3 described above.

To efficiently induce the user to awake at an appropriate time point, the electronic device may obtain an awaking time point of the user in advance. In an embodiment, the electronic device may generate the sleep-inducing music and provide the sleep-inducing music to the user from about an hour before the awakening time point of the user, such that the user may transfer to the first or second NREM state Non REM 1&2 and then transfer to the Wake state.

For example, when the user is in the third NREM sleep state Non REM 3 before a certain time period from the awakening time point, in operation 610, the electronic device may provide the sleep-inducing music to induce the state of the user to the first Non-REM sleep state Non REM 1 or the second Non-REM sleep state Non REM 2, i.e., the target state.

For example, when the user is in the REM sleep state REM before a certain time period from the awaking time, in operation 615, the electronic device may provide sleep-inducing music to induce the state of the user to the first Non-REM sleep state Non REM 1 or the second Non-REM sleep state Non REM 2, i.e., the target state.

In operation 620, when it is determined that the current state of the user changes to the first Non-REM sleep state Non REM 1 or the second Non-REM sleep state Non REM 2, the electronic device may provide the sleep-inducing music to induce the state of the user to the Drowsy state, i.e., the target state in the next stage. Compared with the sleep-inducing music provided in operation 610 or operation 615, the sleep-inducing music provided in operation 620 may have additional melodies, a faster tempo, a faster rhythm, or increased complexity of harmony.

In a process where the sleep-inducing music provided in operation 610 or operation 615 is updated to the sleep-inducing music provided in operation 620, the complexity of harmony may preferentially increase, and then the rhythm may become faster, and last, the melodies may be added, but the embodiment is not limited thereto.

In operation 630, when it is determined that the current state of the user changes to the Drowsy state, the electronic device may provide the sleep-inducing music to induce the state of the user to the Wake state, i.e., the target state in the next stage. Compared with the sleep-inducing music provided in operation 620, the sleep-inducing music provided in operation 630 may have additional melodies, a faster tempo, a faster rhythm, or increased complexity of harmony.

In a process where the sleep-inducing music provided in operation 620 is updated to the sleep-inducing music provided in operation 630, the complexity of harmony may preferentially increase, and then the rhythm may become faster, and last, the melodies may be added, but the embodiment is not limited thereto. Volume may linearly increase for a certain time period while progressing from operation 610 or operation 615 to operation 630.

In an embodiment, when the user is in the first Non-REM sleep state Non REM 1 or the second Non-REM sleep state Non REM 2 before a certain time period from the awaking time, only operation 620 and operation 630 may be performed. Operation 620 and operation 630 may correspond to operation 341 or operation 344 in FIG. 3 described above.

For example, music suitable for inducing to each sleep state may be set as Table 1 below.

TABLE 1

| Sleep stage | Wake | Drowsy | Non-REM 1&2 | Non-REM 3 | . . . |
|---|---|---|---|---|---|
| Tempo | 60~120 BPM | 60~120 BPM | 60~120 BPM | 60~120 BPM | |
| Rhythm | eighth note | quarter note | half note | half note with | |
| | | | | a dot | |
| Harmony | Complexity: | Complexity: | Complexity: | Complexity: | |
| | High | Middle | Low | Low | |
| | Frequency: | Frequency: | Frequency: | Frequency: | |
| | High | Middle | Low | Low | |
| Melody | Tune | Tune change: | Tune change: | Tune change: | |
| | change: High | Middle | Low | Low | |
| | Degree of | Degree of | Degree of | Degree of | |
| | identification | identification | identification | identification | |
| | from an initial | from the initial | from the initial | from the initial | |
| | melody: High | melody: Middle | melody: Low | melody: Low | |
| Volume | V | 0.3 V | 0.09 V | <30 dB | |
| Sound | ◯ | Δ | Δ | Δ | |
| effect | | | | | |

Referring to FIG. 1, when the user is induced from the Wake state to the deep sleep state Non-REM 3, by stages, the rhythm may be slowed down, the harmony may be simplified, the melodies may be simplified (or removed), the volume may be turned down, the sound effects may be removed, and on the other hand, when the user is induced from the deep sleep state Non-REM 3 to the Wake state, by stages, the rhythm may become faster, the harmony may be made more complicated (or added), the volume may be turned up, and the sound effects may be added.

In an embodiment, when external noise occurs while the user sleeps, the sound effects may be added to the sleep-inducing music such that the external noise does not interrupt the sleep of the user. In this case, the sleep-inducing music may make masking against the external noise.

Examples of a scenario to which the operations in FIGS. 4 to 6 are applied are as follows.

### Example 1

To induce user A to sleep onset, music composed of harmony with intermediate complexity, rhythm of eighth notes, and melody matching a genre preferred by the user A is provided in collective consideration of information regarding brainwave (Beta wave), a heart rate, eyeball movements, tosses and turns, and breaths of the user A that just went to bed. After a certain time period, body of the user A begins to gradually loosen, with recovery to regular breaths and gradual comfort in mind, the user A begins to sleep. As the user A begins sleep, a brain wave cycle of the user A gradually slows down, and when the brainwave that has slowed down (Alpha wave) lasts at least for a minute, it is determined that the user A is in a drowsy state. When it is determined that the user A is in the drowsy state, the harmony in the sleep-inducing music is decreased to leap within a third to a fifth, and the sleep-inducing music is converted to music having low complexity, melody with intermediate-low complexity, and quarter-note rhythm. Next, the user A transfers to the first NREM sleep state or the second NREM sleep state, i.e., a deeper sleep state. The brain wave of the user A becomes slow and regular, and muscles of the user A are completely relaxed, and the body of the user A gradually reaches the third NREM sleep state, i.e., a deep sleep state. The brainwave of the user A at an hour before awakening may indicate the first NREM sleep state. After playing the music with low complexity, the melody with intermediate-low complexity, and the quarter-note rhythm for twenty minutes for reaching an appropriate wake-up state thirty minutes before awakening, to provide a sufficient sleep experience, and for fresh awakening, music composed of harmony with intermediate complexity, eighth-note rhythm, and the melody matching the genre preferred by the user A is played for ten minutes to wake the brain wave of the user A. At the awakening time, the user A may spontaneously wake up.

### Example 2

User B who wakes up twice or three times each night and hardly takes deep sleep Although the user B looks like being in the deep sleep state, based examination on a heart rate, a brain wave, eyeball movements, and the like of the user B, it seems that the user B fails to transfer again to the deep sleep state and remains in the REM sleep state. To maintain the sleep state of the user B, the electronic device induces the brain wave of the user B to gradually slow down from an Alpha wave to a Theta wave by providing music composed of low complexity, harmony with intermediate-low complexity, and quarter-note rhythm. Next, as the eyeball movements of the user B slow down and the breaths and the brainwave of the user B gradually slow down, the user B enters the first NREM state or the second NREM state. When the user enters the first NREM state or the second NREM state, the sleep-inducing music composed of half notes and having melody composed of least sounds and harmony with low complexity where a harmony is maintained for a phrase (four nodes) is provided to the user and induces the user to fall into deeper sleep. The user B falls into extremely deep sleep in the third NREM state. The brain wave, which is a Delta wave, becomes extremely slow and regular, and no more eyeball movements are found. The sleep-inducing music is provided with changes to have a rhythm of half notes with a dot and harmony with low complexity where one phrase (four to eight nodes) maintains one harmony and then is removed, such that the deep sleep of the user B may be maintained. At an hour before the awakening, the user B is in the REM sleep state. Music corresponding to the Theta wave is provided to induce the state of the user B an hour before the wake-up to the first Non REM state or the second Non REM state such that the user B does not feel confused after the awakening. Next, after providing music having harmonies with low complexity and intermediate-low complexity and quarter-note rhythm for the user B to emerge to an appropriate wake state before awakening, music composed of harmony with intermediate complexity, eighth-note rhythm, and melody matching the genre preferred by the user is provided for the user B to transfer to the wake state. At the awakening time, the user B may spontaneously wake up.

### Example 3

The user C has hard times waking up from deep sleep and thus is often work for late. At about an hour from awakening, the user C, who is still in the deep sleep state, is in the third Non REM state. For the user C to efficiently awaken, the electronic device provides the user C with sleep-inducing music for inducing gradual wake-up. First, music composed of harmony with low complexity and half-note rhythm is provided to synchronize the brain wave of the user C with a Theta wave, to thereby induce the user C to the first or the second NREM sleep state Non REM 2. Next, after providing the music composed of melody with intermediate-low complexity and a quarter-note rhythm for a certain time period for the user C to spontaneously wake up, music composed of melody with low complexity, eighth-note rhythm, and melody matching the genre preferred by the user is provided such that the user C may transfer to the wake state. At the awakening time, unlike always, the user C may spontaneously wake up.

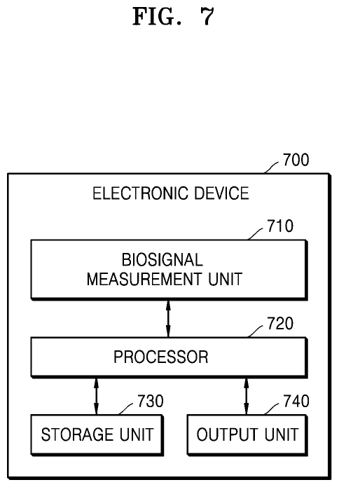

FIG. 7 is a block diagram of an electronic device according to an embodiment of the present disclosure.

The electronic device 700 may be a device configured to obtain a biosignal of the user and generate and provide the sleep-inducing music according to the current sleep state of the user determined based on the obtained biosignal. The electronic device 700 may include, for example, smartphones, tablet personal computers (PC), mobile phones, video phones, e-book readers, desktop PCs, laptop PCs, netbook computers, workstations, servers, personal digital assistants (PDA), portable multimedia players (PMP), mobile medical instruments, cameras, wearable devices, domestic apparatuses, or various computing devices. The electronic device 700 according to an embodiment of the present disclosure is not limited to the aforementioned examples, and the electronic device 700 may include various types of devices configured to generate and provide the sleep-inducing music according to the current sleep state of the user.

Referring to FIG. 7, the electronic device 700 may include a biosignal measuring unit 710, a processor 720, a storage unit 730, and an output unit 740. In an embodiment, the electronic device 700 may also be implemented by more components than the components illustrated in FIG. 7, and the electronic device 700 may also be implemented by more components than the components illustrated in FIG. 7.

In an embodiment, the biosignal measuring unit 710 may include at least one of an EEG sensor, an ECG sensor, a heart rate sensor, an eyeball movement sensor, a PPG sensor, an inertial measurement unit (IMU) (e.g., a gyroscope sensor), a camera, or a microphone. The biosignal measuring unit 710 is not limited to the aforementioned examples, and may be configured in various forms to measure biosignals of various users.

The EEG sensor may include a plurality of electrodes attached to a scalp of the user. The EEG sensor may be configured to record EEG by amplifying delicate electric movements of a brain through the plurality of electrodes attached to the scalp of the user.

The ECG sensor may be configured to measure electric signals generated from a heart of the user, measure heart beats when depolarization and repolarization of heart muscles occur, and express the heart rate in the form of a graph. The ECG sensor may be configured to measure the number of heart beats of the user per unit time.

The eyeball movement sensor may be configured to track eyeball movements of the user. For example, the eyeball movement sensor may be configured to detect movements of muscles around eyes through electrodes, or may be configured to detect movements of eyelids or eyeballs through image data.

The PPG indicates a sensor configured to use light to measure a blood flow rate, i.e., a type of a biosignal. The PPG may be configured to calculate the biosignals by measuring degree of light adsorption by hemoglobin in blood vessels of the user. Through PPG technology, the biosignals such as the number of heart beats or oxygen saturation may be measured or calculated by using light at body parts at which the blood vessels pass. As blood is pumped in the heart, the blood flow rate in each of the blood vessels distributed in the body repeatedly becomes strong and weak in synchronization with pulses, and change in the blood flow rate in microvessels in bio tissues may be detected. For example, through the PPG technology, an event that the blood vessels becomes broad and narrow as the blood flow rate changes as time passes may be measured through properties such as degree of adsorption and transmittance of light.

The IMU may include an accelerometer, an angular velocity meter, and a geomagnetic field sensor, and may be configured to measure complex movements of objects. In an embodiment, the IMU may be attached to the body of the user and measure movements such as tosses and turns of the user.

The camera is a device configured to obtain images, and the electronic device 700 may be configured to obtain biosignals (or biometric data) such as the number of heart beats and breaths of the user by analyzing the images obtained through the camera. For example, the camera may include a Red-Green-Blue (RGB) camera.

The microphone is a device configured to receive sound, and the electronic device 700 may be configured to obtain the biosignals such as the number of heart beats and the number of breaths of the user by analyzing the sound obtained through the microphone.

The storage unit 730 may be configured to store a program to be executed by the processor 720 to be described hereinafter to control operations of the electronic device 700. The storage unit 730 may be configured to store a program including at least one instruction to control the operation of the electronic device 700. Instructions and program codes that may be read by the processor 720 may be stored in the storage unit 730. In an embodiment, the processor 720 may be implemented to execute the instructions or codes of the program stored in the storage unit 730. The storage unit 730 may be configured to data input to the electronic device 700 or output from the electronic device 700.

The storage unit 730 may include at least one type of storage medium among, for example, a flash memory, hard disk, multimedia card micro type, card-type storage unit (e.g., an SD or XD storage unit), random access memory (RAM), static random access memory (SRAM), read-only memory (ROM), electrically erasable programmable read-only memory (EEPROM), programmable read-only memory (PROM), a magnetic storage unit, a magnetic disk, and an optical disk. However, the storage unit is not limited to the aforementioned examples, and may include any type of storage medium in which data may be stored.

Programs stored in the storage unit may be sorted into a plurality of modules according to functions thereof. For example, the programs stored in the storage unit may be sorted into a biosignal identification module, a sleep state identification module, a sleep-inducing music generation module, and the like. The sleep-inducing music generation module may include a database in which pre-generated pieces of music are stored. In an embodiment, the database may also be included in the storage unit 730, separate from the sleep-inducing music generation module.

The output unit 740 may provide the generated sleep-inducing music to the user. For example, the output unit 740 may include at least one of a speaker, an earphone, a headset, or a vibration-type earphone. However, the output unit 740 is not limited to the aforementioned example, and may include various components configured to provide sound to the user.

In an embodiment, the output unit 740 may be configured as a speaker or an output terminal of a headphone. In this case, the electronic device 700 may not include a component configured to output sound and provide information regarding the sleep-inducing music to another output device connected to the electronic device 700 through wireless connection or wired connection such that the other output device provides the sleep-inducing music to the user.

The processor 720 may be configured to control general operations of the electronic device 700. For example, the processor 720 may be configured to generally control the biosignal measurement unit 710, the storage unit 730, the output unit 740, and the like by executing the programs stored in the storage unit 730.

The processor 720 may be configured as a hardware component configured to execute calculation, logic and input/output operation, and signal processing. The processor 720 may be configured to as, for example, at least one of a Central Processing Unit, a microprocessor, a Graphic Processing Unit, Application Specific Integrated Circuits, Digital Signal Processors (DSPs), Digital Signal Processing Devices (DSPDs), Programmable Logic Devices (PLDs), and Field Programmable Gate Arrays), but is not limited thereto.

The processor 720 may be configured to, by executing the at least one instruction stored in the storage unit 730, generate the sleep-inducing music according to the user state determined based on the biosignal of the user and provide the generated sleep-inducing music to the user. For example, the processor 720 may be configured to, by executing the at least one instruction stored in the storage unit 730, obtain the biosignal of the user through the biosignal measurement unit 710, identify the current sleep state of the user based on the obtained biosignal, generate the sleep-inducing music based on the current sleep state of the user, and provide the generated sleep-inducing music to the user through the output unit 740.

The obtaining of the biosignal of the user through the biosignal measurement unit 710 by the processor 720 may correspond to operation 110 in FIG. 1 described above. The identifying of the current sleep state of the user based on the obtained biosignal by the processor 720 may correspond to operation 120 in FIG. 1 described above. The generating of the sleep-inducing music based on the current sleep state of the user by the processor 720 may correspond to operation 130 in FIG. 1 described above. The providing of the generated sleep-inducing music to the user by the processor 720 may correspond to operation 140 in FIG. 1 described above.

In an embodiment, the electronic device 700 may further include a user input receiver. The user input receiver may be, for example, a device with a touch panel. In an embodiment, the electronic device 700 may be configured to obtain at least one of information regarding the target sleep state to use for generating the sleep-inducing music or user information including information regarding the musical preference of the user, through the user input receiver. The user information may include at least one of the musical preference (a player, composition of musical instruments), the tendency, and the emotional state of the user.

As described above, according to an embodiment of the present disclosure, by determining a state of the user through real-time analysis on the biosignal of the user and generating the sleep-inducing music to provide to the user based on the current state of the user, sound suitable for the state of the user may be provided, and the user may be induced to sleep by stages.

In addition, according to an embodiment of the present disclosure, by generating the sleep-inducing music to provide to the user in consideration of the user information such as the musical preference, the genre preference, the tendency, and the emotional state of the user, the sleep-inducing music personalized for the user may be provided, and user experiences may be optimized.

Furthermore, according to an embodiment of the present disclosure, by composing, selecting, or arranging the sleep-inducing music to provide to the user in consideration of various musical factors, natural musical source may be generated, and the sleep state of the user may be efficiently induced.

Various embodiments of the present disclosure may be implemented or supported by one or more computer programs, which may be formed from computer-readable program codes and recoded in computer-readable media. In the present disclosure, “application” and “program” may indicate one or more computer programs, software components, instruction sets, procedures, functions, objects, classes, instances, and related data suitable for implementation in the computer-readable program codes, or a portion thereof. “The computer-readable program codes” may include various types of computer codes including source codes, purpose codes, and executable codes. “The computer-readable medium” may include various types of media that may be accessed by the computer, such as read only memory (ROM), random access memory (ROM), hard disk drive (HDD), compact disc (CD), digital video disc (DVD), or various types of memory devices.

In addition, an apparatus-readable recording medium may be provided in the form of a non-transitory storage medium. Here, ‘the non-transitory storage medium’ is a tangible device, and may exclude wired, wireless, optical, or other communication links configured to transmit temporal electric signals or other signals. The ‘non-transitory storage medium’ does not distinguish a case where the data is semi-permanently stored in the storage medium and a case where the data is temporarily stored in the storage medium. For example, the ‘non-transitory storage medium’ may include a buffer in which the data is temporarily stored. A computer-readable medium may include any available media that may be accessed by a computer and includes all of volatile media, nonvolatile media, removable media, and non-removable media. The computer-readable medium includes a medium in which the data may be permanently stored, and a medium in which data may be stored and overwritten, for example, a rewritable optical disc or an erasable memory device.

According to an embodiment, the method according to various embodiments disclosed herein may be provided in a computer program product. As a merchandise, the computer program product may be provided between sellers and buyers. The computer program product may be distributed in the form of a machine-readable storage medium (e.g., compact disc read only memory (CD-ROM)), or may be directly distributed online (e.g., download or upload) through an application store or between two user devices (e.g., smartphones). In case of online distribution, at least a portion of the computer program product (e.g., a downloadable app) may be at least temporary stored or temporally generated in a machine-readable storage medium such as a server of a manufacturer, a server of an application store, or a memory of an intermediary server.

The descriptions of the present disclosure are to provide examples, and those skilled in the art of the present disclosure may understood that the present disclosure may be easily modified into other specific forms without modification on the technical spirit or indispensable features of the disclosure. Therefore, the embodiments written above will be understood as illustrative in all aspects, not as limitative sense. For example, each of the components described as a single type may be distributed for implementation, and likewise, the components described as distributed may also be combined for implementation.

The scope of the present disclosure may be expressed by the following claims, rather than by the detailed descriptions, and all changes or modifications derived from the meaning, the scope of the claims and equivalents thereof will be construed as being included in the scope of the present disclosure.

1. A method of providing sleep-inducing music by an electronic device, the method comprising:
obtaining a biosignal of a user;
identifying a current sleep state of the user based on the obtained biosignal;
generating the sleep-inducing music based on a current sleep state of the user; and
providing the generated sleep-inducing music to the user.

2. The method of claim 1, wherein
the biosignal of the user comprises at least one of pieces of information regarding electroencephalography, electrocardiogram, a heart rate, eyeball movements, or breath.

3. The method of claim 1, wherein
the current sleep state of the user is identified as wake, drowsy, rapid eye movement (REM) sleep, first non-REM sleep (Non REM 1), second non-REM sleep (Non REM 2), or third non-REM sleep (Non REM 3).

4. The method of claim 1, wherein
the generating of the sleep-inducing music comprises:
identifying a target sleep state of the user; and
generating the sleep-inducing music based on the current sleep state of the user and the target sleep state of the user.

5. The method of claim 1, further comprising
obtaining user information regarding the user,
wherein the generating of the sleep-inducing music comprises generating the sleep-inducing music based on the current sleep state of the user and the user information.

6. The method of claim 1, wherein
the generating of the sleep-inducing music comprises generating the sleep-inducing music based on at least one musical factor among volume, tempo, rhythm, melody, harmony, timbre, tonality, or a number of layers.

7. The method of claim 1, wherein
the generating of the sleep-inducing music comprises composing the sleep-inducing music based on the current sleep state of the user.

8. The method of claim 1, wherein
the generating of the sleep-inducing music comprises selecting music from a database in which pieces of pre-generated music are stored, based on the current sleep state of the user.

9. The method of claim 8, wherein
the generating of the sleep-inducing music further comprises arranging the selected music based on the current sleep state of the user.

10. The method of claim 1, wherein
the generated sleep-inducing music comprises a sound effect.

11. An electronic device configured to provide sleep-inducing music to a user, the electronic device comprising:
a biosignal measurement unit;
a storage unit storing at least one instruction;
at least one processor configured to execute at least one instruction stored in the storage unit; and
an output unit, wherein
the at least one processor is configured to, by executing the at least one instruction:
obtain a biosignal of the user through the biosignal measurement unit;
identify a current sleep state of the user based on the obtained biosignal;
generate the sleep-inducing music based on the current sleep state of the user; and
provide the generated sleep-inducing music to the user through the output unit.

12. The electronic device of claim 11, further comprising a user input receiver, wherein
the at least one processor is configured to:
obtain information regarding a target sleep state of the user through the user input receiver; and
generate the sleep-inducing music based on the current sleep state of the user and the target sleep state of the user.

13. The electronic device of claim 11, further comprising a user input receiver, wherein
the at least one processor is configured to:
obtain user information regarding the user through the user input receiver; and
generate the sleep-inducing music based on the current sleep state of the user and the user information.

14. The electronic device of claim 11, wherein
the storage unit comprises a database in which pieces of pre-generated music are stored, and
the at least one processor is configured to select music from the database, based on the current sleep state of the user, and generate the sleep-inducing music by arranging the selected music based on the current sleep state of the user.

15. A computer-readable recording medium having recorded thereon a program for executing the method of claim 1 on a computer.
